(19) United States
(12) Patent Application Publication
(10) Pub. No.: US 2019/0382810 A1
(43) Pub. Date: Dec. 19, 2019

(54) METHOD FOR PRODUCING LACTIC ACID FROM BIOMASS-BASED MATERIAL

(71) Applicant: Far Eastern New Century Corporation, Taipei City (TW)

(72) Inventors: Chu-Chin Wang, Taipei City (TW); Yu-Chuan Chuang, Taipei City (TW); Shu-Yin Shih, Taipei City (TW); Yi-Cyun Jhang, Taipei City (TW)

(21) Appl. No.: 16/213,150

(22) Filed: Dec. 7, 2018

(30) Foreign Application Priority Data
Jun. 13, 2018 (TW) 107120357

(51) Int. Cl. C12P 7/56; C12N 9/04

(52) U.S. Cl. CPC C12P 7/56; C12Y 101/01027; C12N 9/0006

(57) ABSTRACT

A method for producing lactic acid from a biomass-based material includes: cultivating a strain of Bacillus spp. or recombinant Candida spp. capable of yielding lactic acid by consumption of hexose and pentose in a seed medium containing molasses and corn steep liquor, so as to obtain a seed culture of the strain in the seed medium; and fermenting the biomass-based material with the seed culture of the strain. The biomass contains a fermentable sugar. Genomic DNA of the recombinant Candida spp. includes a gene encoding lactate dehydrogenase, and pdc gene in the genomic DNA of the recombinant Candida spp. is deleted, disrupted or disabled.

### CROSS-REFERENCE TO RELATED APPLICATION

This application claims priority of Taiwanese Invention Patent Application No. 107120357, filed on Jun. 13, 2018.

### FIELD

The disclosure relates to a method for producing lactic acid, and more particularly to a method for producing lactic acid from a biomass-based material.

### BACKGROUND

Lactic acid is a valuable compound, and has been widely applied in food, cosmetic, pharmaceutical and chemical industries. Lactic acid can also be used to synthesize biodegradable plastics. Recently, usage of organic wastes to produce biomass energy is increasingly gaining attention. Cellulosic biomass, a commonly used renewable energy resource, can be converted to lactic acid by virtue of chemical methods and biological methods. Since biological methods are both cost-effective and environmental-friendly, they have been industrially applied to treat organic wastes.

Biological methods are directed to converting cellulosic biomass to lactic acid by virtue of microbial fermentation. Common microorganisms for lactic acid fermentation include lactic acid bacteria (LAB), such as Lactobacillus spp., Lactococcus spp., Pediococcus spp., Enterococcus spp., Streptococcus spp., and Bacilius spp.

In the process of producing lactic acid from a cellulosic biomass, it is necessary to first hydrolyze the cellulosic biomass to obtain a cellulosic hydrolysate. The cellulosic hydrolysate usually contains reducing sugars and fermentation inhibitors (e.g., acetic acid, furfural, hydroxymethyl furfural (HMF), levulinic acid, phenolic compounds, etc.) that are generated by degradation of hemicellulose and reducing sugars during the process. These fermentation inhibitors will inhibit the growth and fermentation of microorganisms, rendering the utilization of reducing sugars less efficient, even being decreased, thereby adversely affecting lactic acid yield.

For decreasing the adverse effects caused by the fermentation inhibitors, previous studies have reported that microorganisms may exhibit improved tolerance to the inhibitors through acclimation, so as to effectively utilize reducing sugars to produce lactic acid. For example, Jiang T. at al. developed an inhibitor-tolerance strain. Bacillus coagulans GKN316, through atmospheric and room temperature plasma (ARTP) mutation and then conducting evolution experiment in condensed dilute-acid hydrolysate (CDH) of corn stover. As shown, the growth and lactic acid yield of the GKN316 strain in the xylose medium containing 25% or 50% CDH are higher than those of the parental strain (Jiang T. et al. (2016), PLoS One, 11 :e0149101).

It is known that yeast is less efficient in fermenting lactic acid, and even not capable of producing lactic acid because of its lack of LDH gene encoding lactate dehydrogenase (LDH). Thus, in recent years, a number of studies have been conducted with the aim to obtain yeast with the ability to produce lactic acid or with improved lactic acid production via genetic engineering. For instance, Ikushima S. et al. constructed a metabolically-engineered Candida utilis Cupdc1Δ4-LDH2 strain that produces L-lactic acid from glucose with excellent efficiency as follows. Initially, the gene encoding pyruvate decarboxylase (CuPDC1) of Candida utilis strain was subjected to four CuPDC1 disruption events to obtain a Cupdc1-null mutant (i.e., Cupdc1Δ4). Two copies of the L-lactate dehydrogenase (L-LDH) gene derived from Bos taurus under the control of the CuPDC1 promoter were then integrated into the genome of the CuPdc1-null mutant to obtain the Cupdc1Δ4 -LDH2 strain (Ikushima S. et al. (2009), Biosci. Biotechnol. Biochem., 73:1818-1824). In a further study, Tamakawa H. et al. developed a genetically-modified pyruvate decarboxylase-deficient Candida utilis strain (mXR/XDH/XK) that expresses an L-lactate dehydrogenase, an NADH-preferring mutated xylose reductase (XR), a xylitol dehydrogenase (XDH) and a xylulokinase (XK). Such Candida utilis strain is capable of co-fermenting hexose and pentose, and thus can effectively increase the production of lactic acid (Tamakawa H. et al. (2012), J. Biosci. Bioeng., 113:73-75).

Many food industry by-products have recycling value because they contain abundant nutrient sources for microorganism growth. It has been reported that a fermentation medium containing molasses and/or corn steep liquor (CSL) can be used to conduct fermentation, so as to improve the proliferation of lactic acid bacteria, thereby increasing lactic acid yield. For instance, in Lee K. B. et al, (2013), Afr. J. Biotechnol., 12:2013-2018, an inoculum of Lactobacillus salivarius L29 (prepared by culturing the same in MRS broth) was incubated in a fermentation medium with specified increments in concentration of both molasses and CSL, and each of the resultant cultures was subjected to viable cell count and determination of lactic acid yield. The result indicated that the optimum concentrations of molasses and CSL facilitating cell growth and high-level lactic acid production were 6% (v/v) and 6% (v/v), respectively.

In spite of the aforesaid prior art, the applicants endeavor to develop a method for efficiently producing lactic acid from a biomass-based material (such as a hydrolysate of cellulosic biomass), particularly by improving the tolerance of a seed culture of fermenting microorganisms to the aforementioned fermentation inhibitors during fermentation, without requirements to remove the fermentation inhibitors therefrom.

### SUMMARY

Therefore, an object of the disclosure is to provide a method for producing lactic acid from a biomass-based material that can alleviate at least one of the drawbacks of the prior art.

According to the disclosure, the method includes cultivating a strain of Bacillus spp. or recombinant Candida spp. capable of yielding lactic acid by consumption of hexose and pentose in a seed medium containing molasses and corn steep liquor, so as to obtain a seed culture of the strain in the seed medium; and fermenting the biomass-based material with the seed culture of the strain. The biomass-based material contains a fermentable sugar. Genomic DNA of the recombinant Candida spp. includes a gene encoding lactate dehydrogenase, and pdc gene in the genomic DNA of the recombinant Candida spp. is deleted, disrupted or disabled.

### BRIEF DESCRIPTION OF THE DRAWINGS

Other features and advantages of the disclosure will become apparent in the following detailed description of the embodiment (s) with reference to the accompanying drawings, of which:

FIG. 1 is a bar diagram showing the lactic acid yields of recombinant Candida utilis in Control Group A1 and Experimental Groups A1 to A5 after fermentation in a fermentation medium with the presence of acetic acid, in which before fermentation, Control Group A1 and each of Experimental Groups A1 to A5 were respectively cultivated in a YPD40 medium and in a seed medium with 3% (v/v) of corn steep liquor and a respective concentration of cane molasses;

FIG. 2 is a bar diagram showing the lactic acid yields of recombinant C. utilis in Control Group A2 and Experimental Groups A6 to A8 after fermentation in a fermentation medium with the presence of acetic acid, in which before fermentation, Control Group A2 and each of Experimental Groups A6 to A8 were respectively cultivated in a YPD40 medium and in a seed medium with 6% (v/v) of cane molasses and a respective concentration of corn steep liquor;

FIGS. 3 to 5 are bar diagrams showing the lactic acid yields of recombinant C. utilis in each Control Group and Experimental Group after fermentation in a fermentation medium respectively containing various concentrations of acetic acid, furfural, and hydroxymethyl furfural (HMF), in which before fermentation, each of Control Groups was cultivated in a YPD40 medium, and each of Experimental Groups was cultivated in a seed, medium with 3% (v/v) of corn steep liquor and 6% (v/v) of cane molasses;

FIG. 6 is a bar diagram showing the lactic acid yields of recombinant Saccharomyces cerevisiae BCRC 920083 in Comparative Control Groups 1 to 3 and Comparative Experimental Groups 1 to 3 after fermentation in a fermentation medium with the presence of the respective one of acetic acid, furfural and HMF, in which before fermentation, each of Control Groups was cultivated in a YPD40 medium, and each of Experimental Groups was cultivated in a seed medium with 3% (v/v) of corn steep liquor and 6% (v/v) of cane molasses;

FIG. 7 is a bar diagram showing the lactic acid yields of recombinant C. utilis in Control Group A15 and Experimental Group A21 after fermentation in a cellulosic hydrolysate of rice straw as the fermentation medium, in which before fermentation. Control Group A15 and Experimental Group A21 were respectively cultivated in a YPD40 medium and a seed medium with 3% (v/v) of corn steep liquor and 6% (v/v) of cane molasses;

FIG. 8 is a bar diagram showing the lactic acid yields of Bacillus coagulans BCRC 910831 in Control Group B1 and Experimental Groups B1 to B6 after fermentation in a fermentation medium with the presence of acetic acid, in which before fermentation, Control Group B1 and each of Experimental Groups B1 to B6 were respectively cultivated in a YPD50 medium and in a seed medium with 7% (v/v) of corn steep liquor and a respective concentration of cane molasses;

FIG. 9 is a bar diagram showing the lactic acid yields of B. coagulans BCRC 910831 in Control Group B2 and Experimental Groups B7 to B12 after fermentation in a fermentation medium with the presence of acetic acid, in which before fermentation, Control Group 32 and each of Experimental Groups B7 to B12 were respectively cultivated in a YPD50 medium and in a seed medium with 6% (v/v) of cane molasses and a respective concentration of corn steep liquor;

FIGS. 10 to 13 are bar diagrams showing the lactic acid yields of B. coagulans BCRC 910831 in each Control Group and Experimental Group after fermentation in a fermentation medium respectively containing various concentrations of acetic acid, furfural, HMF and levulinic acid, in which before fermentation, each of Control Groups was cultivated in a YPD50 medium, and each of Experimental Groups was cultivated, in a seed medium with 7% (v/v) of corn steep liquor and 6% (v/v) of cane molasses; and

FIG. 14 is a bar diagram showing the lactic acid yields of B. coagulans BCRC 910831 in Control Group B19 and Experimental Group B29 after fermentation in the cellulosic hydrolysate of rice straw as the fermentation medium, in which before fermentation, Control Group B19 and Experimental Group B29 were respectively cultivated in a YPD50 medium and a seed medium with 7% (v/v) of corn steep liquor and 6% (v/v) of cane molasses.

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless defined otherwise, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described. For the sake of clarity, the following definitions are used herein.

As used herein, the terms “tolerance” and “resistance” may be used interchangeably, and refer to the capacity of a seed culture of a microorganism to endure or become less responsive to fermentation inhibitor.

Fermentation inhibitors that were usually generated during the process of producing lactic acid from a cellulosic biomass may adversely affecting the fermenting ability of microorganisms and thus the lactic acid yield. In order to alleviate the undesired effects caused by the fermentation inhibitors and thus to increase the lactic acid yield, the applicants endeavored to develop improved methods and found that by cultivating a strain of Bacillus spp. or recombinant Candida spp. capable of co-fermenting hexose and pentose in a seed medium containing molasses and corn steep liquor, the resultant seed culture would exhibit an enhanced resistance to the fermentation inhibitors at the subsequent fermentation stage, thereby effectively improving glucose and xylose utilization rate and thus increasing the lactic acid yield under the fermentation conditions in the presence of the fermentation inhibitors.

Therefore, the present disclosure provides a method for producing lactic acid from a biomass-based material, including: cultivating a strain of Bacillus spp. or recombinant Candida spp. capable of yielding lactic acid by consumption of hexose and pentose in a seed medium containing molasses and corn steep liquor, so as to obtain a seed culture of the strain in the seed medium; and fermenting the biomass-based material containing a fermentable sugar with the seed culture of the strain. The genomic DNA of the recombinant Candida spp. includes a gene encoding lactate dehydrogenase, and pdc gene in the genomic DNA of the recombinant Candida spp. is deleted, disrupted or disabled.

As used herein, the term “Bacillus spp.” is intended to encompass all strains of Bacillus spp. capable of yielding lactic acid by consumption of hexose and pentose. Examples of the strain of Bacillus spp. suitable for use in the method of this disclosure include, but are not limited to, Bacillus coagulans, Bacillus subtilis, Bacillus amyloliquefaciens, Bacillus megaterium. Bacillus methylotrophicus and combinations thereof. In an exemplary embodiment, the strain of Bacillus spp. is Bacillus coagulans.

According to this disclosure, the term “recombinant Candida spp,” is intended to encompass all recombinant strains of Candida spp. capable of yielding lactic acid by consumption of hexose and pentose, the genomic DNA thereof including a gene encoding lactate dehydrogenase, and pdc gene in the genomic DNA of the recombinant Candida spp. being deleted, disrupted or disabled.

As used herein, the term “delete” refers to partial or entire removal of a coding region of a gene.

As used herein, the term “disrupt” refers to performing deletion, insertion or mutation of nucleotide(s) in a gene, so that the gene no longer produces an active protein, or produces a protein with severely reduced activity.

As used herein, the term “disable” refers to inactivating a gene or the protein encoded by the gene so as to force the gene or protein to lose its activity or function.

Examples of the strain of recombinant Candida spp. suitable for use in the method of this disclosure include, but are not limited to, recombinant Candida utilis, recombinant Candida boidinii, recombinant Candida tropicalis, recombinant Candida parapsilosis and combinations thereof. In an exemplary embodiment, the strain of recombinant Candida spp. is recombinant Candida utilis.

According to this disclosure, the genomic DNA of the recombinant Candida spp. (such as recombinant C. utilis) further includes a gene encoding xylose reductase (XR), a gene encoding xylulose kinase (XK) and a gene encoding xylitol dehydrogenase (XDH).

In an embodiment of the disclosure, the recombinant Candida utilis is prepared, by deleting or disrupting pdc gene in the genomic DNA of a strain of Candida utilis (which is deposited in Biosource Collection and Research Center (BCRC) of Food Industry Research and Development Institute (FIRDI) under an accession number BCRC 20325, and is also deposited in American Type Culture Collection (ATCC) under an accession number ATCC 9950), followed by inserting xr gene, xk gene and xdh gene into the genomic DNA thereof.

As used herein, the term “molasses” refers to syrup obtained by removing sucrose crystals from the massecuite during the refinement of sugars from a plant. The type of molasses suitable for use in the disclosure is not particularly limited, and may include various commercially available products. In certain embodiments, the molasses is selected from the group consisting of cane molasses, beet molasses, citrus molasses, corn molasses, and combinations thereof. In an embodiment of the disclosure, the molasses is cane molasses.

As used herein, the term “corn steep liquor” refers to a concentrated liquid obtained by steeping corn in diluted acid during a corn wet-milling process. The type of corn steep liquor suitable for the disclosure is not particularly limited, and may include various commercially available products.

According to this disclosure, when the strain to be cultivated is the recombinant Candida spp., based on the total volume of the seed medium, the molasses may have a concentration ranging from 4% to 6% (v/v), and said corn steep liquor may have a concentration ranging from 1% to 3% (v/v). In an embodiment of this disclosure, based on the total volume of the seed medium, the molasses and the corn steep liquor respectively have a concentration of 6% (v/v) and 3% (v/v) for cultivating the recombinant Candida spp.

According to this disclosure, when the strain to be cultivated is Bacillus spp., based on the total volume of the seed medium, the molasses may have a concentration ranging from 6 to 7% (v/v), and the corn steep liquor may have a concentration ranging from 6.5% to 7.5% (v/v). In an embodiment of this disclosure, based on the total volume of the seed medium, the molasses and the corn steep liquor respectively have a concentration of 6% (v/v) and 7% (v/v) for cultivating the Bacillus spp.

According to the disclosure, the biomass-based material may be obtained from a cellulosic biomass, a hemicellulosic biomass, a lignocellulosic biomass, or combinations thereof. In an embodiment of this disclosure, the biomass-based material is obtained from a cellulosic biomass.

Examples of the cellulosic biomass suitable for use in this disclosure include, but are not limited, to, bioenergy crops, agricultural residues, municipal solid wastes, industrial solid wastes, sludge from paper manufacture, yard wastes, wood wastes, forestry wastes and combinations thereof.

In certain embodiments, the cellulosic biomass is selected from the group consisting of miscanthus, softwood, hardwood, corn cobs, crop residues (such as corn husks), corn stover, grasses, wheat straw, barley straw, hay, rice straw, switchgrass, waste paper, sugarcane bagasse, sorghum plant material, soybean plant material, components obtained from milling of grains, stems, roots, leaves, wood chips, sawdust, shrubs, vegetables, fruits, flowers, and combinations thereof.

According to the disclosure, the biomass-based material may be a cellulosic hydrolysate prepared by performing a hydrolysis treatment (i.e., saccharification) on a cellulosic biomass.

According to the disclosure, before the hydrolysis treatment, the cellulosic biomass may be subjected to a pretreatment. Suitable pretreatment includes, but is not limited to, steam explosion, thermal chemical pretreatment, mechanical treatment (such as pulverization), acid treatment, organosolv, sulfite pretreatment, and combinations thereof.

As used herein, the term “cellulosic hydrolysate”, “lignocellulosic hydrolysate” and “biomass hydrolysate” can be used interchangeably, and refer to products generated from saccharification of the biomass.

In an embodiment of this disclosure, the cellulosic hydrolysate is prepared by performing a dilute acid treatment, steam explosion treatment and saccharification on rice straw in sequence.

According to the disclosure, during the fermenting step, the cellulosic hydrolysate may include at least one fermentation inhibitor selected from the group consisting of acetic acid, furfural, hydroxyl furfural (HMF), levulinic acid and phenolic compounds.

In certain embodiments, during the fermenting step, the cellulosic hydrolysate includes acetic acid that is present in an amount ranging from 1 to 8 g/L, and preferably, from 3 to 7 g/L.

In certain embodiments, during the fermenting step, the cellulosic hydrolysate includes furfural that is present in an amount ranging from 0.5 to 7 g/L, and preferably, from 1 to 6 g/L.

In certain embodiments, during the fermenting step, the cellulosic hydrolysate includes HMF that is present in an amount ranging from 0.5 to 7 g/L, and preferably, from 1 to 6 g/L.

In certain embodiments, during the fermenting step, the cellulosic hydrolysate includes levulinic acid that is present in an amount ranging from 1 to 8 g/L, and preferably, from 3 to 7 g/L.

In certain embodiments, during the fermenting step, the cellulosic hydrolysate includes phenolic compounds that are present in an amount ranging from 0.1 to 5 g/L, and preferably, from 0.5 to 4 g/L.

As used herein, the term “fermentable sugars” refers to simple sugars (e.g., monosaccharides, disaccharides and short oligosaccharides) that can be converted into end products by fermentation with a fermenting microorganism. In certain embodiments, the fermentable sugar includes at least one of hexose and pentose. In an exemplary embodiment, the fermentable sugar includes glucose and xylose.

According to the disclosure, the fermenting step may be conducted under a condition that is substantially absent of molasses and corn steep liquor.

As used herein, the term “substantially absent of” refers to the lack of meaningful quantities of a specifically identified ingredient. Preferably, the fermenting step is conducted under a condition without the ingredient, or under a condition that the amount of the ingredient has no measurable effect on the fermenting step.

The disclosure will be further described by way of the following examples. However, it should be under stood that the following examples are solely intended for the purpose of illustration and should not be construed as limiting the disclosure in practice.

### EXAMPLES

### General Experimental Materials

1. Recombinant Candida utilis

Recombinant Candida utilis used in the following examples was prepared from Candida utilis BCRC 20325 purchased from BCRC of FIRDI (Taiwan) (equivalent to C. utilis ATCC 9950) substantially in accordance with the procedures described in Ikushima S. et al. (2009), supra and Tamakawa H. et al. (2012), supra. Briefly, the pdc gene encoding pyruvate decarboxylase (PDC) of C. utilis BCRC 20325 was deleted and then the L-lactate dehydrogenase (L-LDH) gene derived from bovine was integrated into the genome of the resulting ΔPDC mutant strain. The ΔPDC mutant strain capable of expressing L-lactate dehydrogenase was then subjected to insertion of a gene encoding xylose reductase (XR), a gene encoding xylitol dehydrogenase (XDH) and a gene encoding xylulokinase (XK) into the genomic DNA thereof, thereby obtaining the recombinant Candida utilis capable of yielding lactic acid by consumption of glucose and xylose.

2. Bacillus coagulans BCRC 910831 was deposited in BCRC of FIRDI on Mar. 3, 2018 and deposited in China Center for Type Culture Collection (CCTCC) under an accession number CCTCC M 2018310 on May 28, 2018.
3. A recombinant Saccharomyces cerevisiae strain for comparative purpose in the following examples was obtained from the applicant's previous U.S. Pat. No. 9,382,557 B2, and has been deposited in Deutsche Sammlung von Mikroorganismen and Zellkulturen GmbH (DSMZ) under an accession number DSM 26705, and deposited in BCRC of FIRDI under an accession number BCRC 920083.
4. Cane molasses and corn steep liquor for formulating a seed medium, were respectively purchased from Fonen And Fonher Enterprise Co., Ltd. and Taiwan Sugar Corporation. The cane molasses contains 435 g/L sucrose, 36.5 g/L glucose, and 86 g/L fructose. The corn steep liquor contains 60.88% (w/w) of water, 17.67% (w/w) of crude proteins, 6.49% (w/w) of crude ashes, and 177.94 ppm of sulfur dioxide.
5. Glucose and xylose were purchased from Echo Chemical Co., Ltd,
6. Acetic acid, furfural, hydroxymethyl furfural (HMF), levulinic acid and CeCO₃ were purchased from Sigma-Aldrich.
7. The recipe of the YPD40 and YPD50 media used in the following examples is shown in Table 1.

TABLE 1

| | |
|---|---|
| YPD40 medium | YPD50 medium |

| | |
|---|---|
| Ingredients | Concentration(%, w/v) |

| | | |
|---|---|---|
| Glucose | 4 | 5 |
| Yeast extract | 1 | 1 |
| Peptone | 2 | 2 |

The balance is deionized water.

8. The recipe of the fermentation medium for recombinant C. utilis is shown in Table 2.

TABLE 2

| Ingredients | Concentration(%, w/v) |
|---|---|
| Glucose | 7 |
| Xylose | 3 |
| Acetic acid | 0.7 |
| CaCO₃ | 4 |

The balance is deionized water.

9. The recipe of the fermentation medium for B. coagulans BCRC 910831 is shown in Table 3.

TABLE 3

| Ingredients | Concentration(%, w/v) |
|---|---|
| Glucose | 8.5 |
| Xylose | 2.5 |
| Acetic acid | 0.8 |
| CaCO₃ | 5 |
| Yeast extract | 1 |

The balance is deionized water.

### General Experimental Procedures

1. High performance liquid chromatography (HPLC):

The test samples were subjected to analysis using a high performance liquid Chromatograph (DIONEX Ultimate 3000) equipped with a refractive index (RI) detector (L2400, Hitachi), according to the laboratory analytical procedures (LAPs) developed by National Renewable Energy Laboratory (NREL), USA. The column and operation conditions for HPLC are as follows: Aminex HPX-87H column (BioRad); mobile phase: 5 mM sulfuric acid (in water); flow rate: 0.6 mL/min; sample injection volume: 20 μL; temperature of the column oven: 65° C.; and RI temperature: 45° C.

### Example 1. Effect of Seed Medium (Containing a Respective Concentration of Cane Molasses and 3% (v/v) of Corn Steep Liquor) on Lactic Acid Yield, in Fermentation Media with Acetic Acid for Recombinant Candida utilis

### Experimental Procedures

The recombinant C. utilis divided into six groups including a control group referred to as Control Group A1, and five experimental groups referred to as Experimental Groups A1 to A5. The recombinant C. utilis of Experimental Groups A1 to A5 were respectively inoculated into the seed media (100 mL) as shown in Table 4, and the recombinant C. utilis of Control Group 1 was inoculated into the YFD40 medium (100 mL) as shown in Table 1.

TABLE 4

Concentration(%)(v/v)

| Group | Cane molasses | Corn steep liquor |
|---|---|---|
| Experimental Group A1 | 2 | 3 |
| Experimental Group A2 | 4 | 3 |
| Experimental Group A3 | 5 | 3 |
| Experimental Group A4 | 6 | 3 |
| Experimental Group A5 | 7 | 3 |

The balance is deionized water.

Subsequently, each group was incubated in a constant-temperature shaking incubator (30° C, 150-200 rpm) under aerobic condition for 24 hours to obtain a cell culture having an OD₆₀₀ of up to 20 (about 4.6 g cells/L). The ceil culture was subjected to centrifugation. The cell precipitate thus obtained was washed with sterile water and then suspended with the fermentation medium as shown in Table 2. The resultant cell suspension was used as a seed culture of the recombinant C. utilis.

Then, the seed culture of each group was inoculated into 100 mL of the fermentation medium as shown in Table 2 at a concentration of 4×10⁸ cell/mL, followed by conducting fermentation in the constant-temperature shaking incubator (33° C., 100-150 rpm) under aerobic condition for 72 hours.

Afterwards, the fermentation culture of each group was subjected to centrifugation at 16,800 rpm for 1 minute, The resultant supernatant was subjected to HPLC according to the method as described in the previous “General experimental procedures” section to determine the lactic acid content (g/L) in the supernatant.

Lactic acid yield was calculated using the following formula (I):

A=[B/(C+D)]×100 (I)

where A=lactic acid yield (%)
B=lactic acid content in the supernatant (g/L)
C=glucose content in the fermentation

medium before fermentation (g/L)
D=xylose content in the fermentation medium before fermentation (g/L)

### Results

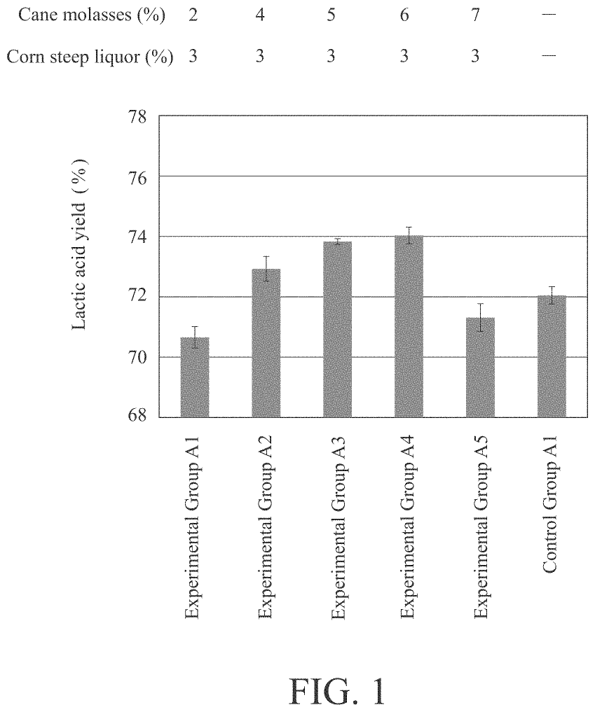

The lactic acid yields of Control Group A1 and Experimental Groups A1 to A5 are shown in FIG. 1. It can be seen from FIG. 1 that the lactic acid yields of Experimental Groups A2 to A4 are higher than that of Control Group A1, The experimental results reveal that the seed culture of the recombinant C. utilis obtained from the seed medium containing 4, 5 or 6% (v/v) of cane molasses and 3% (v/v) of corn steep liquor is capable of effectively utilizing glucose and xylose, thereby enhancing lactic acid yield in the fermentation medium with acetic acid. In particular, the seed medium containing 6% (v/v) of cane molasses and 3% (v/v) of corn steep liquor achieves the highest lactic acid yield.

### Example 2. Effect of Seed Medium (Containing 6% (v/v) of Cane Molasses and a Respective Concentration of Corn Steep Liquor) on Lactic Acid Yield in Fermentation Media with Acetic Acid for Recombinant Candida utilis

### Experimental Procedures

The recombinant C. utilis was divided into four groups including a control group referred to as Control Group A2, and three experimental groups referred to as Experimental Groups A6 to A8. The recombinant C. utilis of Experimental Groups A6 to A8 were respectively inoculated into the seed media (100 mL) as shown in Table 5, and the recombinant C. utilis of Control Group A1 was inoculated into the YPD40 medium (100 mL) as shown in Table 1.

TABLE 5

Concentration (%)(v/v)

| Group | Cane molasses | Corn steep liquor |
|---|---|---|
| Experimental Group A6 | 6 | 1 |
| Experimental Group A7 | 6 | 3 |
| Experimental Group A8 | 6 | 5 |

The balance is deionized water.

Subsequently, each group was subjected to the preparation of the seed culture, fermentation, determination of lactic acid content and calculation of lactic acid yield according to the procedures as described in Example 1.

### Results

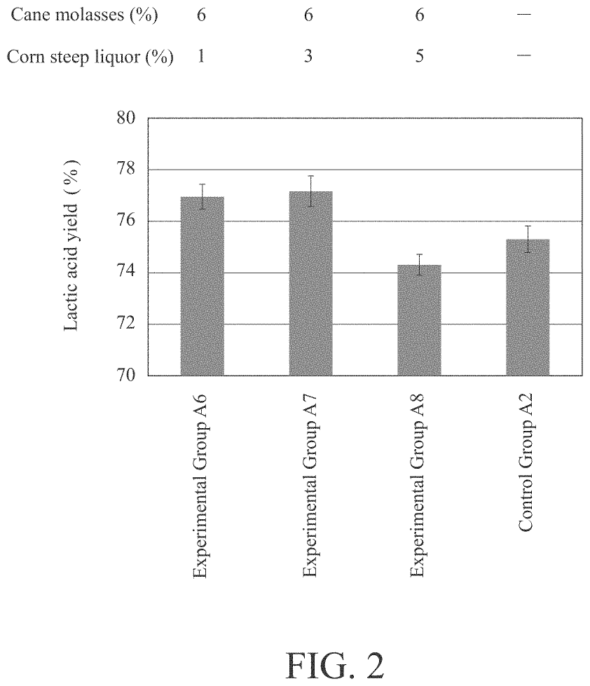

The lactic acid yields of Control Group A2 and Experimental Groups A6 to A8 are shown in FIG. 2. It can be seen from FIG. 2 that the lactic acid yields of Experimental Groups A6 to A7 are higher than that of Control Group A2, indicating that the seed culture of the recombinant C. utilis obtained from the seed medium containing 1 or 3% (v/v) of corn steep liquor and 6% (v/v) of cane molasses is capable of effectively enhancing lactic acid yield in the fermentation medium with acetic acid. In particular, the seed medium containing 3% (v/v) of corn steep liquor and 6% (v/v) of cane molasses achieves the highest lactic acid yield.

### Example 3. Effect of Seed Medium (Containing 3% (v/v) of Corn Steep Liquor and 6% (v/v) of Cane Molasses) on Lactic Acid Yield in Fermentation Media with Various Fermentation Inhibitors for Recombinant Candida utilis

In this example, the fermentation media containing various fermentation inhibitors (including acetic acid, furfural or HMF) were used to mimic a biomass containing fermentation inhibitors (e.g., cellulosic biomass hydrolysate), and the resistance of the seed culture of the recombinant C. utilis that was obtained using the seed medium as mentioned above, to these fermentation inhibitors during fermentation was evaluated.

A. Effect of Acetic Acid on Lactic Acid Yield For Recombinant C. utilis

The recombinant C. utilis was divided into eight groups including four control groups referred to as Control Group A3 to A6, and four experimental groups referred to as Experimental Groups A9 to A12. The recombinant C. utilis of Experimental Groups A9 to A12 were respectively inoculated into the seed medium containing 3% (v/v) of corn steep liquor and 6% (v/v) of cane molasses (100 mL), and the recombinant C. utilis of Control Groups A3 to A6 were respectively inoculated into the YPD40 medium (100 mL) as shown in Table 1.

Subsequently, each group was subjected to the preparation of the seed culture and fermentation in a manner similar to the procedures as described in Example 1, except that each group was incubated under aerobic condition to obtain a cell culture having an OD₆₀₀ of up to 12 (about 2.76 g cells/L) for preparing the seed culture, and the thus obtained seed culture is then inoculated at a concentration of 2.4×10⁸ cells/mL for fermentation. In addition, the fermentation medium for each group is prepared by using the recipe as shown in Table 2, and with different acetic acid concentrations as shown in Table 6.

TABLE 6

| Group | Acetic acid concentration (%)(w/v) |
|---|---|
| Control Group A3 | 0 |
| Experimental Group A9 | |
| Control Group A4 | 0.3 |
| Experimental Group A10 | |
| Control Group A5 | 0.5 |
| Experimental Group A11 | |
| Control Group A6 | 0.7 |
| Experimental Group A12 | |

Thereafter, each group was subjected to determination of lactic acid content and calculation of lactic acid yield according to the procedures as described in Example 1.

### Results

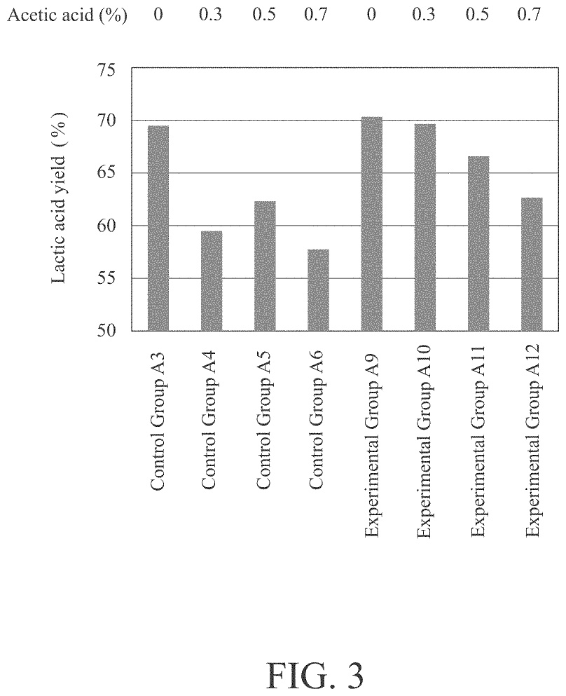

The lactic acid yields of Control Groups A3 to A6 and Experimental Groups A9 to A12 are shown in FIG. 3. It can be seen from FIG. 3 that the lactic yield of each of Experimental Groups A10 to A12 is higher than that of the respective one of Control Groups A4 to A6. The results indicate that the seed culture of the recombinant C. utilis that was obtained from the seed medium containing 3% (v/v) of corn steep liquor and 6% (v/v) of cane molasses is effective in enhancing lactic acid yield under fermentation conditions in the presence of 0.3, 0.5 or 0.7% (w/v) of acetic acid.

B. Effect of Furfural on Lactic Acid Yield for Recombinant C. utilis

The recombinant C. utilis was divided into eight groups including four control groups referred to as Control Group A7 to A10, and four experimental groups referred to as Experimental Groups A13 to A16. Each group was subjected to the preparation of seed culture, fermentation, determination of lactic acid content and calculation of lactic acid yield according to the procedures as described in the abovementioned Section A of this example, except that the acetic acid used in the fermentation medium of each group is respectively replaced with a given concentration of furfural as shown in Table 7.

TABLE 7

| Group | Furfural concentration (%)(w/v) |
|---|---|
| Control Group A7 | 0 |
| Experimental Group A13 | |
| Control Group A8 | 0.1 |
| Experimental Group A14 | |
| Control Group A9 | 0.3 |
| Experimental Group A15 | |
| Control Group A10 | 0.5 |
| Experimental Group A16 | |

### Results

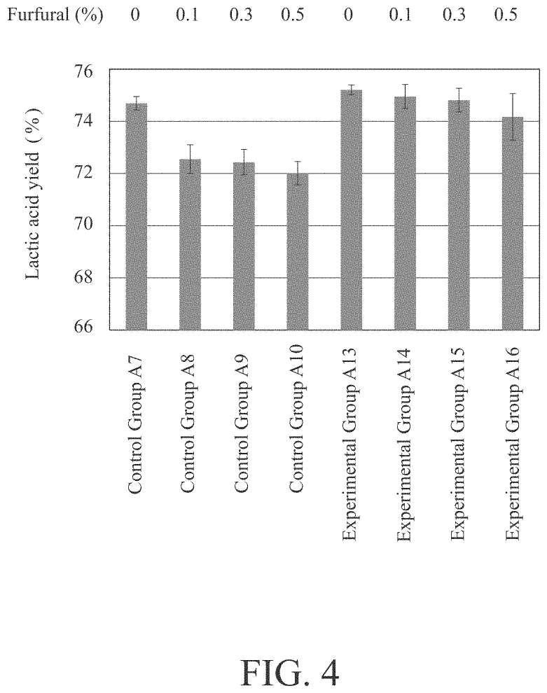

The lactic acid yields of Control Groups A7 to A10 and Experimental Groups A13 to A16 are shown in FIG. 4. It can be seen from FIG. 4 that the lactic acid yield of each of Experimental Groups A14 to A16 is higher than that of the respective one of Control Groups A8 to A10. The results indicate that the seed culture of the recombinant C. utilis that was obtained from the seed medium containing 3% (v/v) of corn steep liquor and 6% (v/v) of cane molasses is effective in enhancing lactic acid yield under fermentation conditions in the presence of 0.1, 0.3 or 0.5 % (w/v) of furfural.

C. Effect of HMF on Lactic Acid Yield for Recombinant C. utilis

The recombinant C. utilis was divided into eight groups including four control groups referred to as Control Group A11 to A14, and four experimental groups referred to as Experimental Groups A17 to A20. Each group was subjected to the preparation of seed culture, fermentation, determination of lactic acid content and calculation of lactic acid yield according to the procedures as described in the abovementioned Section A of this example, except that the acetic acid used in the fermentation medium of each group is respectively replaced with a given concentration of HMF as shown in Table 8.

TABLE 8

| Group | HMF concentration (%)(w/v) |
|---|---|
| Control Group A11 | 0 |
| Experimental Group A17 | |
| Control Group A12 | 0.1 |
| Experimental Group A18 | |
| Control Group A13 | 0.3 |
| Experimental Group A19 | |
| Control Group A14 | 0.5 |
| Experimental Group A20 | |

### Results

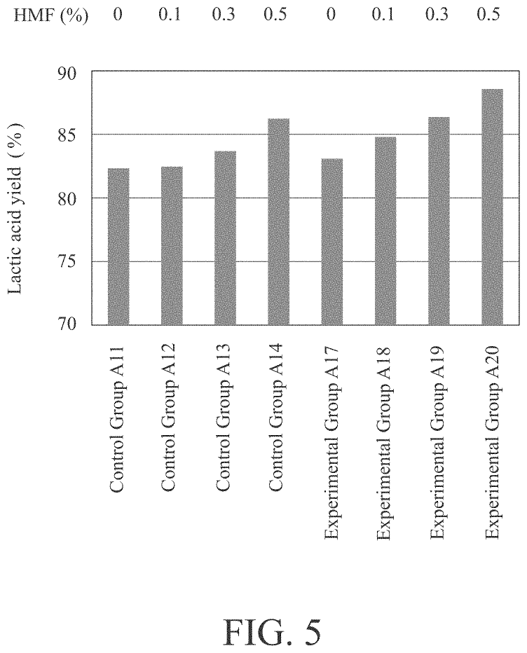

The lactic acid yields of Control Groups A11 to A14 and Experimental Groups A17 to A20 are shown in FIG. 5. It can be seen from FIG. 5 that the lactic acid yield of each of Experimental Groups A18 to A20 is higher than that of the respective one of Control Groups A12 to A14. The results indicate that the seed culture of the recombinant C. utilis that was obtained from the seed medium containing 3% (v/v) of corn steep liquor and 6% (v/v) of cane molasses is effective in enhancing lactic acid yield under fermentation conditions in the presence of 0.1, 0.3 or 0.5 % (w/v) of HMF.

Based on the results of the abovementioned Sections A to C, the applicants deduce that the seed culture of the recombinant C. utilis that was obtained from the seed medium containing 3% (v/v) of corn steep liquor and 6 % (v/v) of cane molasses exhibits an excellent resistance to the fermentation inhibitors (such as acetic acid, furfural and HMF) during fermentation, and thereby being capable of improving glucose and xylose utilisation so as to enhance the lactic acid yield.

D. Effect of Fermentation Inhibitors on Lactic Acid Yield for Recombinant Saccharomyces cerevisiae

In comparison with the recombinant C, utilis used in the abovementioned Sections A to C, the recombinant Saccharomyces cerevisiae BCRC 920083 was subjected to the same analysis to determine whether its seed culture that was obtained from the seed medium containing 3% (v/v) of corn steep liquor and 6% (v/v) of cane molasses, is capable of tolerating the fermentation inhibitors during fermentation.

To be specific, the recombinant S. cerevisiae BCRC 920083 was divided into six groups including three control groups referred to as Comparative Control Groups 1 to 3, and three experimental groups referred to as Comparative Experimental Groups 1 to 3. Each group was subjected to the preparation of seed culture, fermentation, determination of lactic acid content and calculation of lactic acid yield according to the procedures as described in the abovementioned Section A of this example, except that the fermentation inhibitors and their contents in the fermentation medium of each group were as shown in Table 9.

TABLE 9

| Group | Fermentation inhibitor |
|---|---|
| Comparative | 0.3% (w/v) of acetic acid |
| Control Group 1 | |
| Comparative | |
| Experimental Group 1 | |
| Comparative | 0.3% (w/v) of furfural |
| Control Group 2 | |
| Comparative | |
| Experimental Group 2 | |
| Comparative | 0.3% (w/v) of HMF |
| Control Group 3 | |
| Comparative | |
| Experimental Group 3 | |

### Results

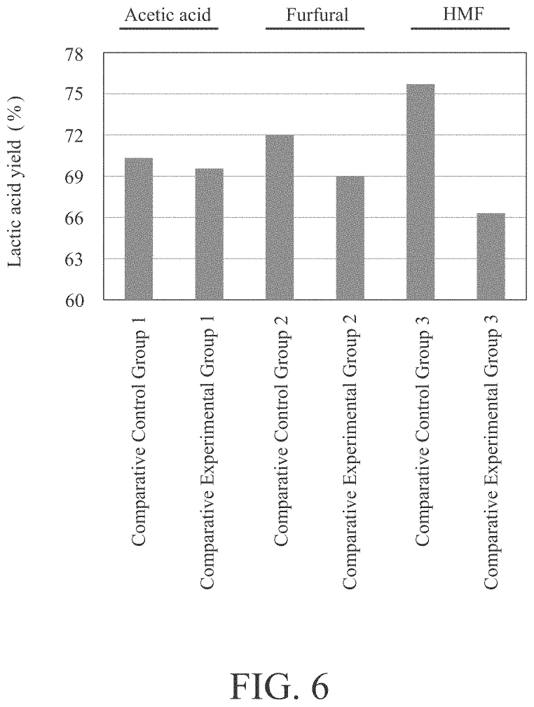

The lactic acid yields of Comparative Control Groups 1 to 3 and Comparative Experimental Groups 1 to 3 are shown in FIG. 6. It can be seen from FIG. 6 that the lactic acid yield of each of Comparative Experimental Groups 1 to 3 is lower than that of the respective one of Comparative Control Groups 1 to 3. The results indicate that the seed culture of the recombinant S. cerevisiae BCRC 920083 obtained from the seed medium containing 3% (v/v) of corn steep liquor and 6% (v/v) of cane molasses cannot tolerate the fermentation inhibitors, rendering it ineffective in utilizing glucose and xylose for lactic acid production.

### Example 4. Effect of Seed Medium (Containing 3% (v/v) of Corn Steep Liquor and 6% (v/v) of Cane Molasses) on Lactic Acid Yield in Cellulosic Hydrolysate of Rice Straw for Recombinant

In this example, a biomass-based material, i.e., a cellulosic hydrolysate of rice straw (which was prepared using a dilute acid as a catalyst via steam explosion), was used as a fermentation medium to investigate the lactic acid yield of the seed culture of the recombinant C. utilis that was obtained from the seed medium containing 3% (v/v) of corn steep liquor and 6% (v/v) of cane molasses.

### Experimental Materials

The rice straw was cut into pieces, followed by pulverization with a pulverizer. The pulverized product of the rice straw was subjected to a dilute acid treatment with a 3% (w/w) sulfuric acid solution, followed by heating at 121° C for 120 to 180 minutes. Then, the resultant mixture was pressed with a vertical press (RESI Co., Ltd.) at a pressure of 1 MPa, so as to obtain a solid portion and a liquid portion. The solid portion thus collected was placed in a steam explosion reactor system (LUCKY SEVEN INDUSTRIAL Co., Ltd.), into which steam was introduced, and was heated at 190 to 200° C. for 3 to 5 minutes. Thereafter, the pressure of the steam explosion reactor system was subjected to a sharp decrease to 1 atm so as to conduct a steam explosion treatment. The pulp thus obtained was mixed with the liquid portion obtained above. The resultant mixture was added with NaOH to adjust the pH value thereof to 5.5, followed by conducting saccharification using an enzymatic mixture (Novozymes Cellic® CTec3 which is composed of cellulase and hemicellulase; dosage: 30 FPU per gram: of cellulosic biomass) under agitation at 70 rpm and 50° C. for 72 hours, in which a 5 N NaOH solution was added at the 2nd, 4th, 8th and 24th hours to maintain the mixture at a pH value of 5.0, so as to obtain a cellulosic hydrolysate of rice straw.

The cellulosic hydrolysate of rice straw was subjected to HPLC according to the method set forth in section 1 of “General Experimental Procedures”, so as to determine the contents of saccharides and fermentation inhibitors. The result shows that the cellulosic hydrolysate of rice straw includes 7% (w/v) of glucose, 1.5% (w/v) of xylose, 0.3% (w/v) of acetic acid, 0.1% (w/v) of furfural, and 0.05% (w/v) of HMF.

### Experimental Procedures

The recombinant C. utilis was divided into two groups including one control group referred to as Control Group A15, and one experimental group referred to as Experimental Group A21. The recombinant C. utilis of Experimental Group A21 and Control Group A15 were respectively inoculated, into the seed medium containing 3% (v/v) of corn steep liquor and 6% (v/v) of cane molasses (100 mL), and the YPD40 medium (100 mL) as shown in Table 1.

Subsequently, each group was subjected to the preparation of seed culture, fermentation, determination of lactic acid content and calculation of lactic acid yield in a manner similar to the procedures as described in Example 1, except that the cellulosic hydrolysate of rice straw was used as the fermentation medium, and the period of the fermentation is 48 hours.

### Results

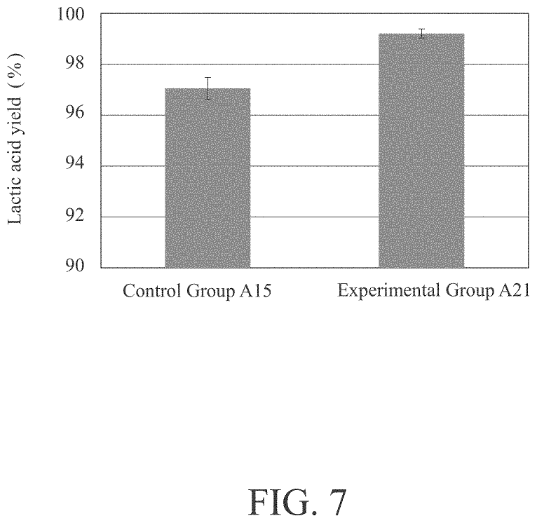

The lactic acid yields of Control Group A15 and Experimental Group A21 are shown in FIG. 7. It can be seen from FIG. 7 that the lactic yield of Experimental Group A21 is higher than that of Control Group A15, indicating that the seed culture of the recombinant C. utilis obtained from the seed medium containing 3% (v/v) of corn steep liquor and 6% (v/v) of cane molasses is capable of effectively utilizing glucose and xylose present in the cellulosic hydrolysate of rice straw during fermentation, thereby enhancing lactic acid yield.

### Example 5. Effect of Seed Medium (Containing Cane Molasses at a Respective Concentration and 7% (v/v) of Corn Steep Liquor) on Lactic Acid Yield in Fermentation Media with Acetic Acid for Bacillus coagulans

### Experimental Procedures

B. coagulans BCRC 910831 was divided into seven groups including a control group referred to as Control Group B1, and six experimental groups referred to as Experimental Groups B1 to B6. Experimental Groups B1 to B6 were respectively inoculated into the seed media (100 mL) as shown in Table 10, and Control Group B1 was inoculated into the YPD50 medium (100 mL) as shown in Table 1.

TABLE 10

Concentration(%)(v/v)

| Group | Cane molasses | Corn steep liquor |
|---|---|---|
| Experimental Group B1 | 3 | 7 |
| Experimental Group B2 | 4 | 7 |
| Experimental Group B3 | 5 | 7 |
| Experimental Group B4 | 6 | 7 |
| Experimental Group B5 | 7 | 7 |
| Experimental Group B6 | 8 | 7 |

The balance is deionized water.

Subsequently, each group was incubated in a constant-temperature shaking incubator (50° C. 150 rpm) under aerobic condition for 24 hours to obtain a cell culture having an OD₄₂₀ of up to 8 (about 0.8 g cells/L). The cell culture was subjected to centrifugation and the thus obtained cell precipitate was washed with sterile water and then suspended with the fermentation medium as shown in Table 3. The resultant cell suspension was used as a seed culture of B. coagulans BCRC 910831.

Then, the seed culture of each group was inoculated into 100 mL of the fermentation medium as shown in Table 3 at a concentration of 4×10⁹ cell/mL, followed by fermentation in a constant-temperature shaking incubator (50° C., 150 rpm) under anaerobic condition for 48 hours. The resulting fermentation product of each group was subjected to determination of lactic acid content and calculation of lactic acid yield according to the procedures as described in Example 1.

### Results

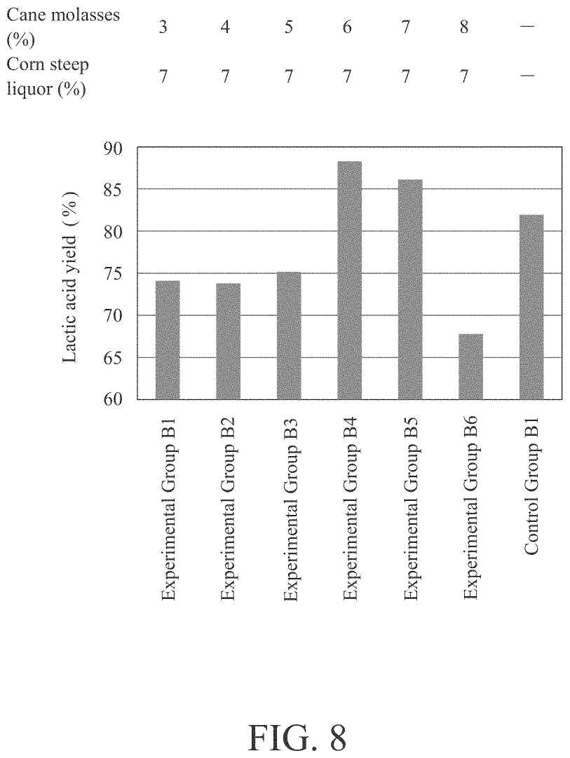

The lactic acid yields of Control Group B1 and Experimental Groups B1 to B6 are shown in FIG. 8. It can be seen from FIG. 8 that the lactic acid yields of Experimental Groups 34 and B5 are higher than that of Control Group B1, indicating that the seed culture of B. coagulans BCRC 910831 obtained from the seed medium containing 6 or 7% (v/v) of cane molasses and 7% (v/v) of corn steep liquor is capable of effectively utilising glucose and xylose, thereby enhancing lactic acid yield in the fermentation medium with acetic acid. In particular, the seed medium containing 6% (v/v) of cane molasses and 7% (v/v) of corn steep liquor achieves the highest lactic acid yield.

### Example 6. Effect of Seed Medium (Containing 6% (v/v) of Cane Molasses and Corn Steep Liquor at a Respective Concentration) on Lactic Acid Yield in Fermentation Media with Acetic Acid for Bacillus coagulans

### Experimental Procedures

B. coagulans BCRC 910831 was divided into seven groups including a control group referred to as Control Group 32, and six experimental groups refer red to as Experimental Groups B7 to B12. Experimental Groups B7 to B12 were respectively inoculated into the seed media (100 mL) as shown in Table 11, and Control Group B2 was inoculated into the YPD50 medium (100 mL) as shown in Table 1.

TABLE 11

Concentration(%)(v/v)

| Group | Cane molasses | Corn steep liquor |
|---|---|---|
| Experimental Group B7 | 6 | 3 |
| Experimental Group B8 | 6 | 4 |
| Experimental Group B9 | 6 | 5 |
| Experimental Group B10 | 6 | 6 |
| Experimental Group B11 | 6 | 7 |
| Experimental Group B12 | 6 | 8 |

The balance is deionized water.

Subsequently, each group was subjected to the preparation of seed culture, fermentation, determination of lactic acid and calculation of lactic acid yield in a manner similar to the procedures as described in Example 6, except that the period of the fermentation is 72 hours.

### Results

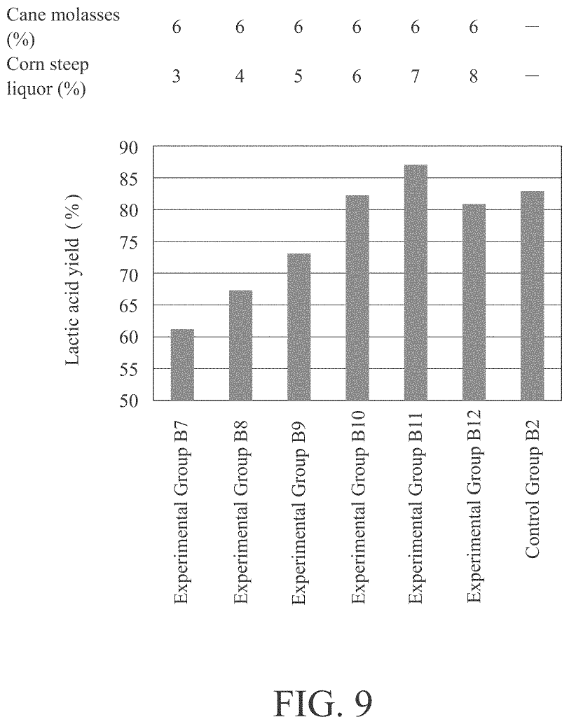

The lactic acid yields of Control Group 32 and Experimental Groups B7 to B12 are shown in FIG. 9. It can be seen from FIG. 9 that the lactic acid yield of Experimental Group B11 is higher than that of Control Group B2, indicating that the seed culture of B. coagulans BCRC 910831 obtained from the seed medium containing 6% (v/v) of cane molasses and 7% (v/v) of corn steep liquor is capable of enhancing lactic acid yield in the fermentation medium with acetic acid.

### Example 7. Effect of Seed Medium (Containing 7% (v/v) of Corn Steep Liquor and 6% (v/v) of Cane Molasses) on Lactic Acid Yield in Fermentation Media with Various Fermentation Inhibitors for Bacillus coagulans

In this example, the fermentation media containing various fermentation inhibitors (including acetic acid, furfural, HMF or levulinic acid) were used to mimic a biomass-based material containing fermentation inhibitors (e.g., cellulosic biomass hydrolysate), and the resistance of the seed culture of B. coagulans BCRC 910831 obtained using the seed medium as mentioned above, to these fermentation inhibitors during fermentation was evaluated.

A. Effect on Acetic Acid on Lactic Acid Yield for B. coagulans BCRC 910831

B. coagulans BCRC 910831 was divided into eight groups including four control groups referred to as Control Group B3 to B6, and four experimental groups referred to as Experimental Groups B13 to B16. Experimental Groups B13 to B16 were respectively inoculated into the seed medium containing 7% (v/v) of corn steep liquor and 6% (v/v) of cane molasses (100 mL), and Control Groups B3 to B6 were respectively inoculated in to the YPD50 medium (100 mL) as shown in Table 1.

Subsequently, each group was subjected to the preparation of seed culture and fermentation in a manner similar to the procedures as described in Example 5, except that the period of the fermentation is 72 hours. In addition, the fermentation medium for each group is prepared by using: the recipe as shewn in Table 2 and with different concentrations of acetic acid as shown in Table 12.

TABLE 12

| Group | Acetic acid concentration (%)(w/v) |
|---|---|
| Control Group B3 | 0 |
| Experimental Group B13 | |
| Control Group B4 | 0.3 |
| Experimental Group B14 | |
| Control Group B5 | 0.5 |
| Experimental Group B15 | |
| Control Group B6 | 0.7 |
| Experimental Group B16 | |

Thereafter, each group was subjected to determination of lactic acid content and calculation of lactic acid yield according to the procedures as described in Example 1.

### Results

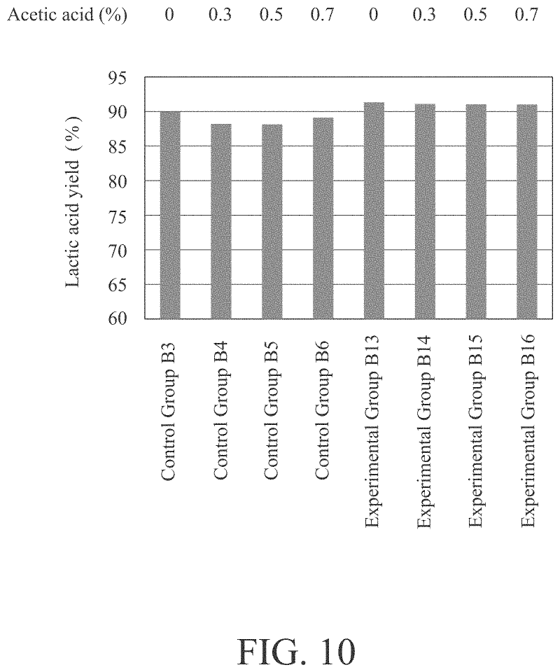

The lactic acid yields of Control Groups B3 to B6 and Experimental Groups B13 to B16 are shown in FIG. 10. It can be seen from FIG. 10 that the lactic acid yield of each of Experimental Groups B14 to A16 is higher than that of the respective one of Control Groups B4 to B6. The results indicate that the seed culture of B. coagulans BCRC 910831 obtained from the seed medium containing 7% (v/v) of corn steep liquor and 6% (v/v) of cane molasses is effective in enhancing lactic acid yield under fermentation conditions in the presence of 0.3, 0.5 or 0.7% (w/v) of acetic acid.

B. Effect of Furfural on Lactic Acid Yield for B. coagulans BCRC 910831

B. coagulans BCRC 910831 was divided into eight groups including four control groups referred to as Control Group B7 to B10, and four experimental groups referred to as Experimental Groups B17 to B20. Each group was subjected to the preparation of seed culture, fermentation, determination of lactic acid content and calculation of lactic acid yield according to the procedures as described in the abovementioned Section A of this example, except that the acetic acid used in the fermentation medium for each group is respectively replaced with a given concentration of furfural as shown in Table 13.

TABLE 13

| Group | Furfural concentration (%)(w/v) |
|---|---|
| Control Group B7 | 0 |
| Experimental Group B17 | |
| Control Group B8 | 0.2 |
| Experimental Group A18 | |
| Control Group B9 | 0.4 |
| Experimental Group A19 | |
| Control Group B10 | 0.6 |
| Experimental Group B20 | |

### Results

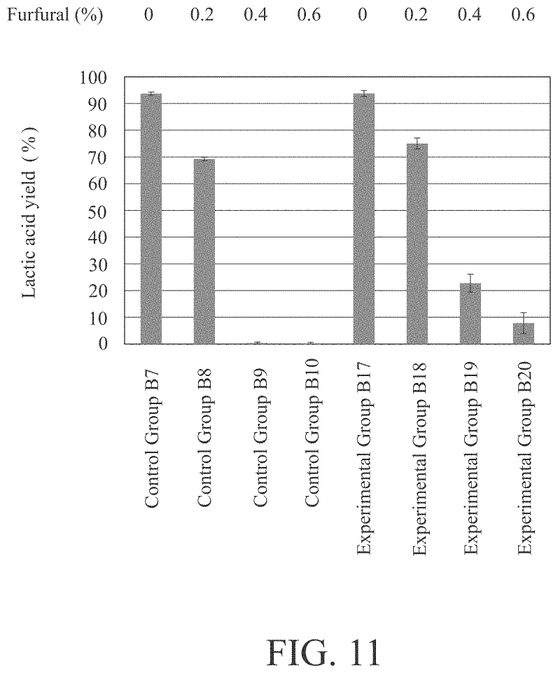

The lactic acid yields of Control Groups B7 to B10 and Experimental Groups B17 to B20 are shown in FIG. 11. It can be seen from FIG. 11 that the lactic acid yield of each of Experimental Groups B18 to B20 is higher than that of the re spec: five one of Control Groups B8 to B10. The results indicate that the seed culture of B. coagulans BCRC 910831 obtained from the seed medium containing 7% (v/v) of corn steep liquor and 6% (v/v) of cane molasses is effective in enhancing lactic acid yield under fermentation conditions in the presence of 0.2, 0.4 or 0.6 % (w/v) of furfural.

C. Effect of HMF on lactic acid yield for B. coagulans BCRC 910831

B. coagulans BCRC 910831 was divided into eight groups including four control groups referred to as Control Group B11 to B14, and four experimental groups referred to as Experimental Groups B21 to B24. Each group was subjected to the preparation of seed culture, fermentation, determination of lactic acid content and calculation of lactic acid yield according to the procedures as described in the abovementioned Section A of this example, except chat the acetic acid used in the fermentation medium for each group is respectively replaced with a given concentration of HMF as shown in Table 14.

TABLE 14

| Group | HMF concentration (%)(w/v) |
|---|---|
| Control Group B11 | 0 |
| Experimental Group B21 | |
| Control Group B12 | 0.2 |
| Experimental Group B22 | |
| Control Group B13 | 0.4 |
| Experimental Group B23 | |
| Control Group B14 | 0.6 |
| Experimental Group B24 | |

### Results

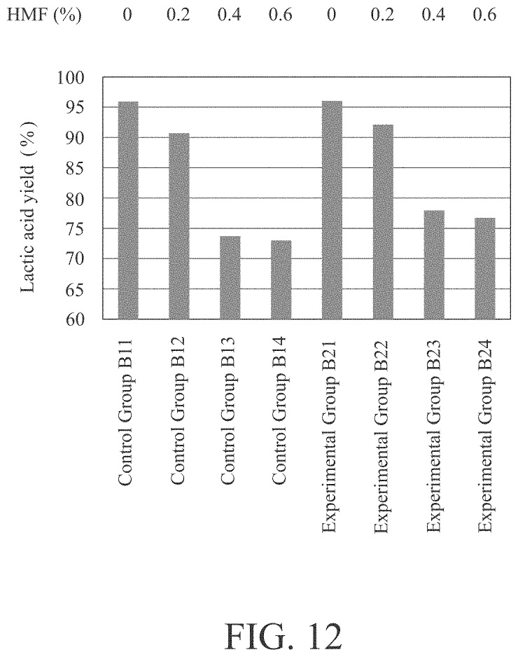

The lactic acid yields of Control Groups B11 to B14 and Experimental Groups B21 to B24 are shown in FIG. 12. It can be seen from FIG. 12 that the lactic yield of each of Experimental Groups B22 to B24 is higher than that of the respective one of Control Groups B12 to B14. The results indicate that the seed culture of B. coagulans BCRC 910831 obtained from the seed medium containing 7% (v/v) of corn steep liquor and 6% (v/v) of cane molasses is effective in enhancing lactic acid yield under fermentation conditions in the presence of 0.2, 0.4 or 0.6% (w/v) of HMF.

D. Effect of Levulinic Acid on Lactic Acid Yield for B. coagulans BCRC 910831

B. coagulans BCRC 910831 was divided into eight groups including four control groups referred to as Control Group B15 to B18, and four experimental groups referred to as Experimental Groups B25 to B28. Each group was subjected to the preparation of seed culture, fermentation, determination of lactic acid content and calculation of lactic acid yield according to the procedures as described in the abovementioned Section A of this example, except that the acetic acid used in the fermentation medium for each group is respectively replaced with a given concentration of levulinic acid as shown in Table 15.

TABLE 15

| Group | Levulinic acid concentration (%)(w/v) |
|---|---|
| Control Group B15 | 0 |
| Experimental Group B25 | |
| Control Group B16 | 0.3 |
| Experimental Group B26 | |
| Control Group B17 | 0.2 |
| Experimental Group B27 | |
| Control Group B18 | 0.7 |
| Experimental Group B28 | |

### Results

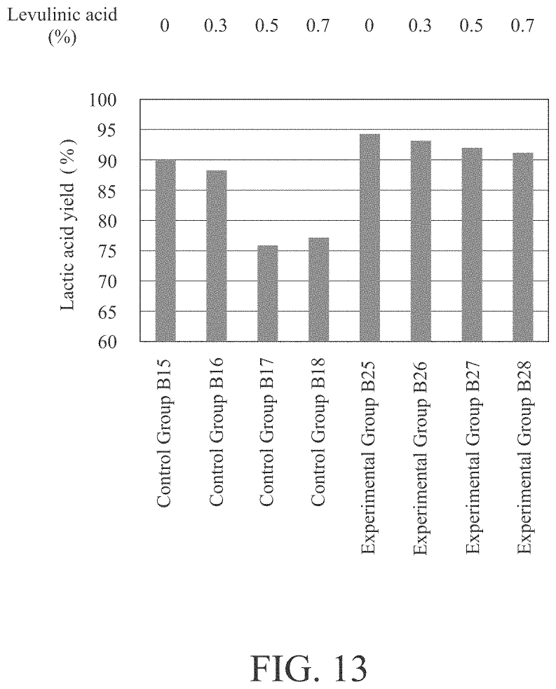

The lactic acid yields of Control Groups B15 to B18 and Experimental Groups B25 to B28 are shown in FIG. 13. It can be seen from FIG. 13 that the lactic acid yield of each of Experimental Groups B26 to B28 is higher than that of the respective one of Control Groups B16 to B18. The results indicate that the seed culture of B. coagulans BCRC 910831 obtained from the seed medium, containing 7% (v/v) of corn steep liquor and 6% (v/v) of cane molasses is effective in enhancing lactic acid yield under fermentation conditions in the presence of 0.3, 0.5 or 0.7 % (w/v) of levulinic acid.

Based on the results of the abovementioned Sections A to D, the applicants deduce that the seed culture of B. coagulans BCRC 910831 obtained from the seed medium containing 7% (v/v) of corn steep liquor and 6% (v/v) of cane molasses exhibits an excellent resistance to the fermentation inhibitors (such as acetic acid, furfural, HMF and levulinic acid) during fermentation, thereby being capable of improving glucose and xylose utilization, so as to enhance the lactic acid yield.

### Example 8. Effect of Seed Medium (Containing 7% (v/v) of Corn Steep Liquor and 6% (v/v) of Cane Molasses) on Lactic Acid Yield in Cellulosic Hydrolysate of Rice Straw for Bacillus coagulans

In this example, the cellulosic hydrolysate prepared in Example 5 was used as a fermentation medium to investigate the lactic acid yield of the seed culture of B. coagulans BCRC 910831 obtained from the seed medium containing 7% (v/v) of corn steep liquor and 6% (v/v) of cane molasses.

### Experimental Procedures

B. coagulans BCRC 910831 was divided into two groups including one control group referred to as Control Group B19, and one experimental group referred to as Experimental Groups B29. B. coagulans BCRC 910831 of Experimental Group B29 and Control Group B19 were respectively inoculated, into the seed medium containing 7% (v/v) of corn steep liquor and 6% (v/v) of cane molasses (100 mL), and the YPD50 medium (100 mL) as shown in Table 1.

Subsequently, each group was subjected to the preparation of seed culture, fermentation, determination of lactic acid content and calculation of lactic acid yield in a manner similar to the procedures as described in Example 5, except that the cellulosic hydrolysate of rice straw was used as the fermentation medium.

### Results

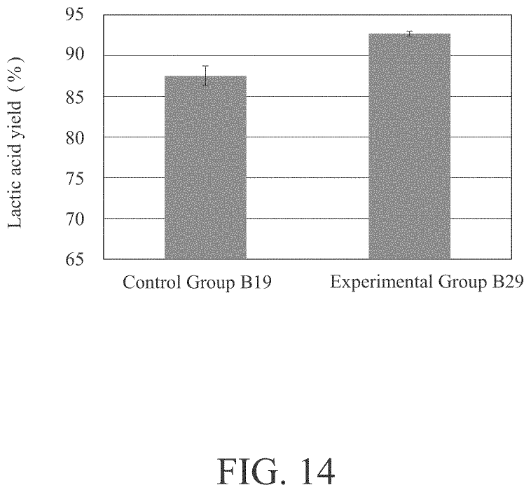

The lactic acid yields of Control Group B19 and Experimental Group B29 are shown in FIG. 14. It can be seen from FIG. 14 that the lactic acid yield of Experimental Group B29 is higher than that of Control Group B19, indicating that the seed culture of B. coagulans BCRC 910831 obtained from, the seed medium, containing 7% (v/v) of corn steep liquor and 6% (v/v) of cane molasses is capable of effectively utilizing glucose and xylose pre sent in the cellulosic: hydrolysate of rice straw for fermentation, thereby enhancing the lactic acid yield.

In view of the foregoing, by cultivation of Bacillus spp. or recombinant Candida spp. capable of co-fermenting hexose (such as glucose) and pentose (such as xylose) in the seed medium containing cane molasses and corn steep liquor, the resulting seed culture is shown to exhibit an excellent resistance to the fermentation inhibitors that may be generated during fermentation, and thus is capable of effectively utilizing glucose and xylose to enhance the lactic acid yield under fermentation conditions in the presence of fermentation inhibitors.

All patents and references cited in this specification are incorporated herein in their entirety as reference. Where there is conflict, the descriptions in this case, including the definitions, shall prevail.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A method for producing lactic acid from a biomass-based material, comprising:
cultivating a strain of Bacillus spp. or recombinant Candida spp. capable of yielding lactic acid by consumption of hexose and pentose in a seed medium containing molasses and corn steep liquor, so as to obtain a seed culture of the strain in the seed medium; and
fermenting the biomass-based material with the seed culture of the strain;
wherein the biomass-based material contains a fermentable sugar, and
wherein genomic DNA of the recombinant Candida spp. includes a gene encoding lactate dehydrogenase, and pdc gene in the genomic DNA of the recombinant Candida spp. is deleted, disrupted or disabled.

2 . The method as claimed in claim 1, wherein the strain is recombinant Candida spp., and, based on the total volume of the seed medium, the molasses has a concentration ranging from 4% to 6% (v/v), and the corn steep liquor has a concentration ranging from 1% to 3% (v/v).

3. The method as claimed in claim 1, wherein the strain is Bacillus spp., and, based on the total volume of the seed medium, the molasses has a concentration ranging from 6 to 7% (v/v), and the corn steep liquor has a concentration ranging from 6.5% to 7.5% (v/v).

4. The method as claimed in claim 1, wherein the recombinant Candida spp. is selected from the group consisting of recombinant Candida utilis, recombinant Candida boidinii, recombinant Candida tropicalis, recombinant Candida parapsilosis and combinations thereof.

5. The method as claimed in claim 4, wherein the recombinant Candida spp, is recombinant Candida utilis.

6. The method a s claimed in claim 5, wherein the genomic DNA of the recombinant Candida utilis includes a gene encoding xylose reductase, a gene encoding xylulokinase and a gene encoding xylitol dehydrogenase.

7. The method of claim 3, wherein the Bacillus spp. is selected from the group consisting of Bacillus coagulans, Bacillus subtilis, Bacillus amyloliquefaciens, Bacillus megaterium, Bacillus methylotrophicus and combinations thereof.

8. The method as claimed in claim 7, wherein the Bacillus spp. is Bacillus coagulans.

9. The method as claimed in claim 7, wherein the fermenting step is conducted under a condition that is substantially absent of molasses and corn steep liquor.

10. The method as claimed in claim 1, wherein the fermentable sugar includes hexose and pentose.

11. The method as claimed in claim 10, wherein the fermentable sugar includes glucose and xylose.

12. The method as claimed in claim 1, wherein the biomass-based material is a cellulosic hydrolysate.

13. The method as claimed in claim 12, wherein during the fermenting step, the cellulosic hydrolysate includes at least one fermentation inhibitor selected from the group consisting of acetic acid, furfural, hydroxyl furfural (HMF), levulinic acid and phenolic compounds.
